(19) United States
(12) Patent Application Publication
(10) Pub. No.: US 2026/0069822 A1
(43) Pub. Date: Mar. 12, 2026

(54) MEDICAL DEVICE

(71) Applicant: ASAHI INTECC CO., LTD., Seto-shi (JP)

(72) Inventors: Makoto NISHIGISHI, Seto-shi, Aichi (JP); Hirotomo SHIMIZU, Seto-shi, Aichi (JP); Yoshiki KATO, Seto-shi, Aichi (JP); Yusuke KIDO, Seto-shi, Aichi (JP)

(73) Assignee: ASAHI INTECC CO., LTD., Seto-shi (JP)

(21) Appl. No.: 19/382,331

(22) Filed: Nov. 7, 2025

Related U.S. Application Data
(63) Continuation of application No. PCT/JP2023/018774, filed on May 19, 2023.

(51) Int. Cl. A61M 25/00; A61M 25/09

(52) U.S. Cl. CPC A61M 25/0009; A61M 25/09; A61M 2025/0037

(57) ABSTRACT

The medical device is provided with: a first tube having a melting point of a first temperature; a second tube having a melting point of a second temperature, which is different from the first temperature, and whose distal end is positioned further toward a proximal end side than a proximal end of the first tube; and a connection member having a melting point of a third temperature, which is higher than the lower of the first temperature and the second temperature. The connection member is in contact with each of the first tube and the second tube, and indirectly connects the first tube and the second tube while maintaining a separation between the proximal end of the first tube and the distal end of the second tube. The connection member provides a continuous path between the first tube and the second tube.

### CROSS-REFERENCE TO RELATED APPLICATION

The present application claims priority to PCT/JP2023/018774, filed on May 19, 2023, the entire contents of which being incorporated herein by reference.

### TECHNICAL FIELD

The present disclosure relates to a medical device.

### BACKGROUND ART

In order to improve safety and operability, catheters generally have a structure that is flexible on the distal end side and highly rigid on the proximal end side. For example, Patent Literature 1 discloses a method of manufacturing a catheter tube formed by joining, in an axial direction, a distal end side tube having relatively low rigidity and a proximal end side tube having relatively high rigidity. In the method described in Patent Literature 1, the distal end portion of the proximal end side tube is inserted into the proximal end portion of the distal end side tube, and an inner surface of the proximal end portion of the distal end side tube and an outer surface of the distal end portion of the proximal end side tube are joined.

### CITATION LIST

### Patent Literature

Patent Literature 1: JP 2013-005976 A

### SUMMARY

### Technical Problems

However, in the technique described in Patent Literature 1, because the structure has the distal end portion of the proximal end side tube entering the inside of the distal end side tube, a step is formed in the catheter inner cavity. In this case, depending on the type of additional device that is inserted into the catheter (for example, a workhorse wire, a therapeutic device, or a sensor) or the shaping applied to the catheter, the additional device may get caught on the step in the catheter inner cavity. The catching of the additional device in the catheter inner cavity poses the problem of hindering the procedure, and is not preferable in that damage to the catheter may occur.

Note that such a problem is also common in cases where an internal burr (a step or projection) occurs at a connecting part between one tube and another tube, even if the structure does not involve one tube entering the inside of another tube. Furthermore, such a problem is not limited to PCI procedures, and is generally shared by medical devices used in percutaneous procedures. In addition, such a problem is not limited to the vascular system, and is common to medical devices inserted into various organs within the human body, such the lymphatic system, biliary system, urinary tract system, respiratory system, digestive system, secretory glands, and reproductive organs.

The present disclosure has been made to solve at least part of the problem described above, and is directed to suppressing the occurrence of an internal burr at a connecting part between two tubes arranged in the axial direction.

### Solutions to Problems

The present disclosure has been made to solve at least part of the problem described above and others, and can be realized as the following aspects.

(1) According to an aspect of the present disclosure, a medical device is provided. The medical device comprises: a first tube whose melting point is a first temperature; a second tube whose melting point is a second temperature, which is different from the first temperature, and whose distal end is positioned further toward a proximal end side than a proximal end of the first tube; and a connection member whose melting point is a third temperature, which is higher than the lower of the first temperature and the second temperature, the connection member being in contact with each of the first tube and the second tube, and connecting the first tube and the second tube in a separated state.

Note that the present disclosure can be implemented in various forms, for example, in the form of a medical device, a medical tube, a catheter, and manufacturing methods of the same, and the like.

### BRIEF DESCRIPTION OF DRAWINGS

FIG. 1 is an explanatory diagram illustrating a configuration of a catheter serving as a medical device.

FIG. 2 is an explanatory diagram illustrating a configuration of a catheter serving as a medical device.

FIGS. 3A to 3F are transverse cross-sectional views of the catheter.

FIG. 4 is a diagram describing a method of using the catheter.

FIG. 5 is a diagram describing a method of using the catheter.

FIG. 6 is an enlarged longitudinal cross-sectional view of a part of a sensor tube.

FIG. 7 is an enlarged longitudinal cross-sectional view of a part of a sensor tube according to a comparative example.

FIG. 8 is an enlarged longitudinal cross-sectional view of a part of a sensor tube according to a second embodiment.

FIG. 9 is an enlarged longitudinal cross-sectional view of a part of a sensor tube according to a third embodiment.

FIG. 10 is an enlarged longitudinal cross-sectional view of a part of a sensor tube according to a fourth embodiment.

FIG. 11 is an enlarged longitudinal cross-sectional view of a part of a sensor tube according to a fifth embodiment.

FIG. 12 is an explanatory diagram illustrating a configuration of a catheter according to a sixth embodiment.

FIG. 13 is an explanatory diagram illustrating a configuration of a catheter according to a seventh embodiment.

### DETAILED DESCRIPTION

### First Embodiment

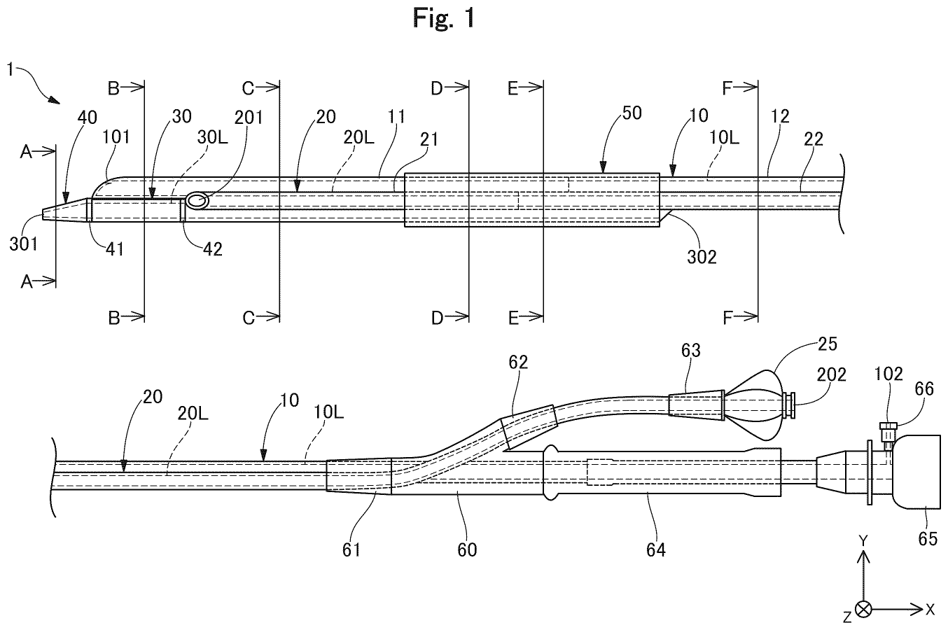

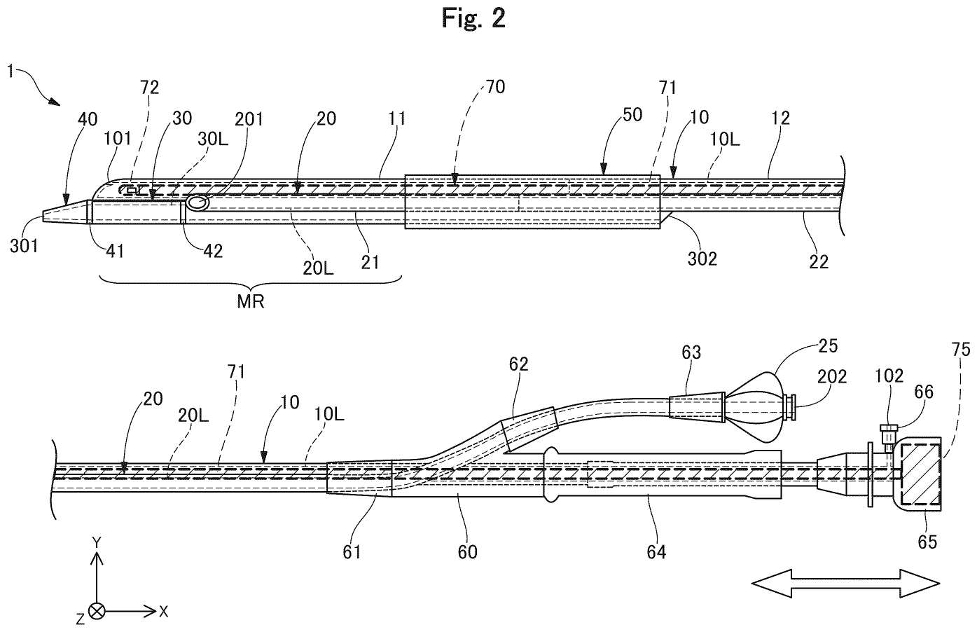

FIGS. 1 and 2 are explanatory diagrams illustrating the configuration of a medical device 1. The medical device 1 according to the present embodiment is a catheter used for treating a lesion in a biological lumen, such as a CTO that has occurred in a blood vessel. Hereinafter, the medical device 1 is also referred to as “catheter 1”. As shown in FIGS. 1 and 2, the catheter 1 includes a sensor tube 10, an OTW (Over The Wire) tube 20, an RX (Rapid Exchange) tube 30, a distal end tip 40, a first marker 41, a second marker 42, a covering portion 50, a branched connector 60, a first reinforcing member 61 to a third reinforcing member 63, a cylindrical member 64, a connector 65, a connector 25, and a sensor 70. The sensor tube 10 is a medical tube. The sensor tube 10 is a medical device. The OTW tube 20 is a medical tube. The OTW tube 20 is a medical device.

In FIG. 1, illustration of the sensor 70 is omitted in order to describe the configuration of the tube and the lumen inside the tube. In FIG. 2, the sensor 70, which is built into a sensor lumen 10L inside the sensor tube 10, is illustrated by a dashed line with diagonal hatching.

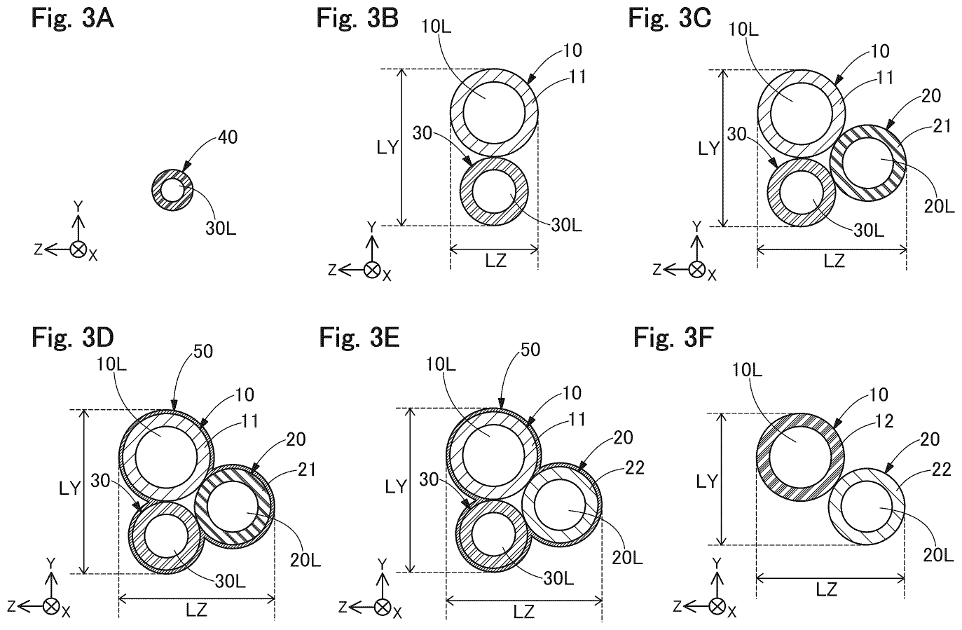

For convenience of the description, FIGS. 1 and 2 include parts where the components are illustrated with a relative size ratio which is different from the actual relative size ratio. Also, portions in which the components are exaggeratedly illustrated may be included. Furthermore, FIGS. 1 and 2 show mutually orthogonal XYZ axes. The X-axis corresponds to the longitudinal direction of the catheter 1, the Y-axis corresponds to the height direction of the catheter 1, and the Z-axis corresponds to the width direction of the catheter 1. The left side (negative X-axis direction) in FIGS. 1 and 2 is referred to as the “distal end side” of the catheter 1 and the components thereof, and the right side (positive X-axis direction) in FIGS. 1 and 2 is referred to as the “proximal end side” of the catheter 1 and the components thereof. Also, of the two ends in the longitudinal direction (X-axis direction) of the catheter 1 and the components thereof, one end positioned on the distal end side is referred to as the “distal end”, and the other end positioned on the proximal end side is referred to as the “proximal end”. In addition, the distal end and the vicinity thereof is referred to as a “distal end portion”, and the proximal end and the vicinity thereof is referred to as the “proximal end portion”. The distal end side is inserted into the living body, and the proximal end side is operated by an operator, such as a physician. These points are also common to FIGS. 3A to 3F and subsequent drawings.

FIGS. 3A to 3F are transverse cross-sectional views of the catheter. FIG. 3A shows a transverse cross-section of the catheter 1 along line A-A in FIG. 1. FIG. 3B shows a transverse cross-section of the catheter 1 along line B-B in FIG. 1. FIG. 3C shows a transverse cross-section of the catheter 1 along line C-C in FIG. 1. FIG. 3D shows a transverse cross-section of the catheter 1 along line D-D in FIG. 1. FIG. 3E shows a transverse cross-section of the catheter 1 along line E-E in FIG. 1. FIG. 3F shows a transverse cross-section of the catheter 1 along line F-F in FIG. 1. Hereinafter, the configuration of the catheter 1 will be described using FIGS. 1, 2, and 3A to 3F.

The sensor tube 10 is a cylindrically-shaped member (tubular body) having an elongated outer shape. The sensor tube 10 extends linearly along the longitudinal direction (X-axis direction) of the catheter 1, parallel to the OTW tube 20 and the RX tube 30. Inside the sensor tube 10, the sensor lumen 10L (dashed line) for accommodating the sensor 70 is formed. The sensor lumen 10L is a lumen for the sensor 70.

The distal end of the sensor tube 10 is positioned at the same position as the distal end of the RX tube 30 in the longitudinal direction of the catheter 1, or a position slightly toward the proximal end side. A distal end opening 101 is formed in the distal end of the sensor tube 10, communicating the distal end of the sensor lumen 10L with the outside. The distal end opening 101 is a fluid discharge port for keeping the inside of the sensor lumen 10L in a wet state. The proximal end of the sensor tube 10 is positioned further toward the proximal end side than the proximal end of the OTW lumen 20L and the proximal end of the RX tube 30 in the longitudinal direction of the catheter 1. On the proximal end side of the sensor tube 10, the first reinforcing member 61, the branched connector 60, the cylindrical member 64, and the connector 65 are attached in this order from the distal end side toward the proximal end side. The details will be described later. A fluid supply portion 66 is attached to the connector 65, and a proximal end opening 102 is formed in the fluid supply portion 66, communicating the proximal end of the sensor lumen 10L with the outside. The proximal end opening 102 is a fluid supply port to the sensor lumen 10L.

As shown in FIG. 1, the sensor tube 10 has a distal end side tube 11 that is disposed on the distal end side, and a proximal end side tube 12 that is disposed further toward the proximal end side than the distal end side tube 11. The distal end side tube 11 and the proximal end side tube 12 are both cylindrical members (tubular bodies) having an elongated outer shape. The distal end side tube 11 and the proximal end side tube 12 are connected at an arbitrary location where the covering portion 50 is provided in the longitudinal direction. That is, the distal end side tube 11 and the proximal end side tube 12 each constitute a portion of the sensor lumen 10L.

The OTW tube 20 is a cylindrically-shaped member (tubular body) having an elongated outer shape. On the distal end side of the branched connector 60, the OTW tube 20 extends linearly along the longitudinal direction of the catheter 1, parallel to the sensor tube 10 and the RX tube 30. Inside the OTW tube 20, an OTW lumen 20L (dashed line) for accommodating a therapeutic device (for example, a plasma guidewire or a penetration guidewire) is formed. The OTW lumen 20L is a so-called over-the-wire (OTW) type lumen that does not have a proximal end opening in the part placed inside the biological lumen during use of the catheter 1.

The distal end of the OTW tube 20 is positioned further toward the proximal end side than the distal end of the sensor tube 10 and the distal end of the RX tube 30 in the longitudinal direction of the catheter 1. A distal end opening 201 is formed in the distal end of the OTW tube 20, communicating the distal end of the OTW lumen 20L with the outside. The distal end opening 201 is a device protrusion port for causing the therapeutic device to protrude toward biological tissue. As a result of the distal end portion of the OTW tube 20 being diagonally cut, the distal end opening 201 faces a direction intersecting the longitudinal direction of the catheter 1. As a result, during use of the catheter 1, it becomes easier to make the therapeutic device reach the biological tissue that is present around the catheter 1. The proximal end of the OTW tube 20 is positioned further toward the distal end side than the proximal end of the sensor tube 10, and further toward the proximal end side than the proximal end of the RX tube 30 in the longitudinal direction of the catheter 1. On the proximal end side of the OTW tube 20, the first reinforcing member 61, the branched connector 60, the second reinforcing member 62, the third reinforcing member 63, and the connector 25 are attached in this order from the distal end side toward the proximal end side. The details will be described later. A proximal end opening 202 is formed in the connector 25, communicating the proximal end of the OTW lumen 20L with the outside. The proximal end opening 202 is a device insertion port for inserting the therapeutic device into the OTW lumen 20L.

As shown in FIG. 1, the OTW tube 20 has a distal end side tube 21 that is disposed on the distal end side, and a proximal end side tube 22 that is disposed further toward the proximal end side than the distal end side tube 21. The distal end side tube 21 and the proximal end side tube 22 are both cylindrical members (tubular bodies) having an elongated outer shape. The distal end side tube 21 and the proximal end side tube 22 are connected at an arbitrary location where the covering portion 50 is provided in the longitudinal direction. That is, the distal end side tube 21 and the proximal end side tube 22 each constitute a portion of the OTW lumen 20L.

The RX tube 30 is a cylindrically-shaped member (tubular body) having an elongated outer shape. The RX tube 30 extends linearly along the longitudinal direction of the catheter 1, parallel to the sensor tube 10 and the OTW tube 20. Inside the RX tube 30, an RX lumen 30L (dashed line) for accommodating a workhorse wire is formed.

The distal end of the RX tube 30 is positioned at the same position as the distal end of the sensor tube 10 in the longitudinal direction of the catheter 1, or a position slightly toward the distal end side. A hollow distal end tip 40 is joined to the distal end portion of the RX tube 30. A distal end opening 301 is formed in the distal end of the distal end tip 40, communicating the distal end of the RX lumen 30L with the outside. The distal end opening 301 is a wire insertion port for inserting the workhorse wire into the RX lumen 30L. The proximal end of the RX tube 30 is positioned further toward the distal end side than the proximal end of the sensor tube 10 and the proximal end of the OTW tube 20 in the longitudinal direction of the catheter 1. A proximal end opening 302 is formed in the proximal end of the RX tube 30, communicating the proximal end of the RX lumen 30L with the outside. The proximal end opening 302 is a wire withdrawal port for withdrawing the workhorse wire to the outside. As a result of the proximal end of the RX tube 30 being diagonally cut, the proximal end opening 302 faces a direction intersecting the longitudinal direction of the catheter 1. As a result, during use of the catheter 1, it becomes easier to withdraw the workhorse wire from the proximal end opening 302.

The distal end tip 40 is a cylindrical member that is radiopaque and has an outer diameter that increases from the distal end side toward the proximal end side. As a result of being joined to the distal end portion of the RX tube 30, the distal end tip 40 is positioned at the distal end of the catheter 1, and advances through a biological lumen ahead of the other members. The inner cavity of the distal end tip 40 communicates with the RX lumen 30L of the RX tube 30, and as described above, the distal end opening 301, which communicates the distal end of the RX lumen 30L with the outside, is formed in the distal end of the distal end tip 40.

The first marker 41 and the second marker 42 are annular members having radiopacity. The first marker 41 is disposed at a position adjacent to the proximal end of the distal end tip 40 on the outer peripheral surface of the RX tube 30, and is joined to the outer peripheral surface of the RX tube 30. The second marker 42 is disposed at a position adjacent to the distal end of the distal end opening 201 of the OTW tube 20 on the outer peripheral surface of the RX tube 30, and is joined to the outer peripheral surface of the RX tube 30. The joining of the first marker 41 and the second marker 42, for example, can be performed by joining resins to each other by heat fusion, or by joining using an adhesive such as an epoxy-based adhesive. The first marker 41 and the second marker 42 may be colored such that the markers can be directly observed by an operator. In this way, by disposing the first marker 41 and the second marker 42 on the RX tube 30, it is possible to suppress interference in the sensing (acquisition of image information) by the sensor 70 caused by the first marker 41 or the second marker 42.

As shown in FIG. 3A, only the distal end tip 40 is present in the transverse cross-section along line A-A. As shown in FIG. 3B, in the transverse cross-section along line B-B, the sensor tube 10 (specifically, the distal end side tube 11) and the RX tube 30 have the outer peripheral surfaces joined to each other. As shown in FIG. 3C, in the transverse cross-section along line C-C, the sensor tube 10 (specifically, the distal end side tube 11), the OTW tube 20 (specifically, the distal end side tube 21), and the RX tube 30 have the outer peripheral surfaces joined to each other. As shown in FIG. 3D, in the transverse cross-section along line D-D, the sensor tube 10 (specifically, the distal end side tube 11), the OTW tube 20 (specifically, the distal end side tube 21), and the RX tube 30 have the outer peripheral surfaces joined to each other. Furthermore, in the D-D transverse cross-section, the covering portion 50 fixes the three tubes 10, 20, and 30 by covering the part of the surface of the three tubes 10, 20, and 30 facing the outer side along the outer periphery of the three tubes 10, 20, and 30. As shown in FIG. 3E, in the transverse cross-section along line E-E, the sensor tube 10 (specifically, the distal end side tube 11), the OTW tube 20 (specifically, the proximal end side tube 22), and the RX tube 30 have the outer peripheral surfaces joined to each other. Similarly, in the E-E transverse cross-section, the covering portion 50 fixes the three tubes 10, 20, and 30 by covering the part of the surface of the three tubes 10, 20, and 30 facing the outer side along the outer periphery of the three tubes 10, 20, and 30. As shown in FIG. 3F, in the transverse cross-section along line F-F, the sensor tube 10 (specifically, the proximal end side tube 12) and the OTW tube 20 (specifically, the proximal end side tube 22) have the outer peripheral surfaces joined to each other.

The joining of the sensor tube 10, the OTW tube 20, and the RX tube 30 may be performed using an arbitrary joining agent such as an epoxy-based adhesive, or by thermal welding. In the B-B transverse cross-section, the C-C transverse cross-section, the D-D transverse cross-section, and the E-E transverse cross-section, the height LY of the catheter 1 is larger than the width LZ of the catheter 1. On the other hand, in the F-F transverse cross-section, the height LY of the catheter 1 is smaller than the width LZ of the catheter 1. Note that, as shown in FIGS. 3A to 3F, the magnitude relationship between the outer diameters of the three tubes 10, 20, and 30 is as follows: outer diameter of sensor tube 10>outer diameter of OTW tube 20>outer diameter of RX tube 30. Furthermore, the magnitude relationship between the inner diameters (lumens) of the three tubes 10, 20, and 30 is as follows: inner diameter of sensor lumen 10L>inner diameter of OTW lumen 20L>inner diameter of RX lumen 30L. However, these magnitude relationships of the outer diameters and inner diameters are merely an example, and may be arbitrarily changed.

The description will be continued returning to FIG. 1. The covering portion 50 is a resin layer for fixing the three tubes 10, 20, and 30 (specifically, the sensor tube 10, the OTW tube 20, and the RX tube 30). The covering portion 50 is provided in a section where the three tubes 10, 20, and 30 extend side-by-side, or in other words, in a section further toward the proximal end side than the distal end opening 201, and further toward the distal end side than the proximal end opening 302. Note that, as shown in FIG. 1, the covering portion 50 may be provided further toward the proximal end side than the distal end opening 201, or in other words, at a position separated from the distal end opening 201 toward the proximal end side. This can suppress interference in the sensing (acquisition of image information) by the sensor 70, which has been inserted in the sensor lumen 10L, caused by the covering portion 50. As shown in FIGS. 3D and 3E, the covering portion 50 thinly covers the parts of the three tubes 10, 20, and 30 facing the outer side along the outer periphery of the three tubes 10, 20, and 30. As a result, the covering portion 50 is capable of fixing the three tubes 10, 20, and 30 while maintaining the recesses (concave portions) formed at the parts adjacent to each of the tubes 10, 20, and 30 in the transverse cross-section of the catheter 1.

The branched connector 60 is a member having an inner cavity that branches into two branches, and is disposed on the proximal end side of the catheter 1. The OTW tube 20 is inserted through one inner cavity of the branched connector 60. The sensor tube 10 is inserted through another inner cavity of the branched connector 60. The first reinforcing member 61 is a cylindrical member disposed further toward the distal end side than the branched connector 60. The first reinforcing member 61 covers the outer periphery of each of the sensor tube 10 and the OTW tube 20 that have been inserted into the branched connector 60, which reinforces the distal end side of the branched connector 60.

The second reinforcing member 62 is a cylindrical member disposed further toward the proximal end side of the one branch of the branched connector 60. The second reinforcing member 62 covers the outer periphery of the OTW tube 20 that has been inserted into the branched connector 60, which reinforces the proximal end side of the branched connector 60. The third reinforcing member 63 is a cylindrical member disposed further toward the distal end side than the connector 25. The third reinforcing member 63 covers the outer periphery of the OTW tube 20 that has been inserted into the connector 25, which reinforces the distal end side of the connector 25. The connector 25 is a member that is joined to the proximal end portion of the OTW tube 20. The connector 25 has a pair of wing portions for an operator to grip. The proximal end opening 202 (device insertion port) is formed at the proximal end of the connector 25, communicating the proximal end of the OTW lumen 20L with the outside.

The cylindrical member 64 is a cylindrical member disposed further toward the proximal end side of the other branch of the branched connector 60. The cylindrical member 64 covers the outer periphery of the sensor tube 10 that has been inserted into the branched connector 60, which reinforces the proximal end side of the branched connector 60. The connector 65 is a member that is joined to the proximal end portion of the sensor tube 10. A housing for accommodating a connection terminal 75 of the sensor 70 is provided on the proximal end side of the connector 65. On the outer peripheral surface of the connector 65, a fluid supply portion 66 is provided, in which a proximal end opening 102 that communicates the proximal end of the sensor lumen 10L with the outside is formed.

The sensor 70 (FIG. 2) is an imaging sensor for acquiring image information. As shown in FIG. 2, the sensor 70 includes a main body portion 71, a probe 72, and a connection terminal 75. The main body portion 71 is an elongated member that extends along the longitudinal direction of the catheter 1. A driving cable (coaxial cable) that electrically connects the probe 72 and the connection terminal 75 is built into the inside of the main body portion 71. The probe 72 includes an ultrasonic transducer (also referred to as an ultrasonic vibrator, a piezoelectric body, an ultrasonic transmission/reception element, or an ultrasonic element) that transmits ultrasonic waves toward biological tissue, and receives the ultrasonic waves that have propagated through the biological tissue and have then been reflected. The probe 72 is also referred to as an imaging core or a transducer. The connection terminal 75 is a terminal that electrically connects the sensor 70 to a console terminal provided externally. The connection terminal 75 is provided at the proximal end of the main body portion 71, and is accommodated inside the housing of the connector 65.

The sensor 70 is electrically connected to an external console terminal via the connection terminal 75, receives a supply of power from the console terminal, and outputs detection signals from the probe 72 to the console terminal. As a result, the console terminal is capable of displaying image information based on the detection signals from the probe 72. As shown in FIG. 2, the sensor 70 is fixed to the connector 65. Furthermore, as indicated by the white arrows in FIG. 2, by gripping and sliding the connector 65 and in the front-rear direction (the direction of the white arrow), an operator is capable of moving the position of the probe 72 of the sensor 70 within a range MR from the distal end of the sensor lumen 10L to the distal end of the covering portion 50, or in other words, within a predetermined range MR that includes the distal end opening 201.

The distal end side tube 11 of the sensor tube 10, the distal end side tube 21 of the OTW tube 20, the RX tube 30, and the covering portion 50 can be formed of a flexible material such as a thermoplastic resin such as polyethylene resin, polypropylene resin, or polyurethane, polyvinyl chloride, ethylene-vinyl acetate copolymer, cross-linked ethylene-vinyl acetate copolymer, polyamide elastomer, polyolefin elastomer, polyurethane elastomer, silicone rubber, or latex rubber. The distal end side tube 11 of the sensor tube 10, the distal end side tube 21 of the OTW tube 20, the RX tube 30, and the covering portion 50 may be formed of the same material, or formed of different materials.

The distal end side tube 11 of the sensor tube 10 and the proximal end side tube 22 of the OTW tube 20 can be formed of a resin having high rigidity such as nylon resin, polyester resin, or PEEK resin. The distal end side tube 11 of the sensor tube 10 and the proximal end side tube 22 of the OTW tube 20 may be formed of the same material, or formed of different materials. Note that any one or more of the distal end side tube 11 and the proximal end side tube 12 of the sensor tube 10, the distal end side tube 21 and the proximal end side tube 22 of the OTW tube 20, and the RX tube 30 may have a multi-layer configuration in which tubes of different materials are layered.

The distal end tip 40, the first marker 41, and the second marker 42 can be formed of a radiopaque resin material or metal material. For example, in a case where a radiopaque resin is used, the distal end tip 40, the first marker 41, and the second marker 42 can be formed by mixing a radiopaque material such as bismuth trioxide, tungsten, or barium sulfate with a polyamide resin, a polyolefin resin, a polyester resin, a polyurethane resin, a silicon resin, a fluororesin, or the like. For example, when a radiopaque metallic material is used, the distal end tip 40, the first marker 41, and the second marker 42 can be formed of gold, platinum, tungsten, or an alloy containing these elements (such as a platinum-nickel alloy). The distal end tip 40, the first marker 41, and the second marker 42 may be formed of the same material, or formed of different materials.

The branched connector 60, the first reinforcing member 61 to the third reinforcing member 63, the cylindrical member 64, the connector 65, and the connector 25 can be formed of a known resin material. The branched connector 60, the first reinforcing member 61 to the third reinforcing member 63, the cylindrical member 64, the connector 65, and the connector 25 may be formed of the same material, or formed of different materials.

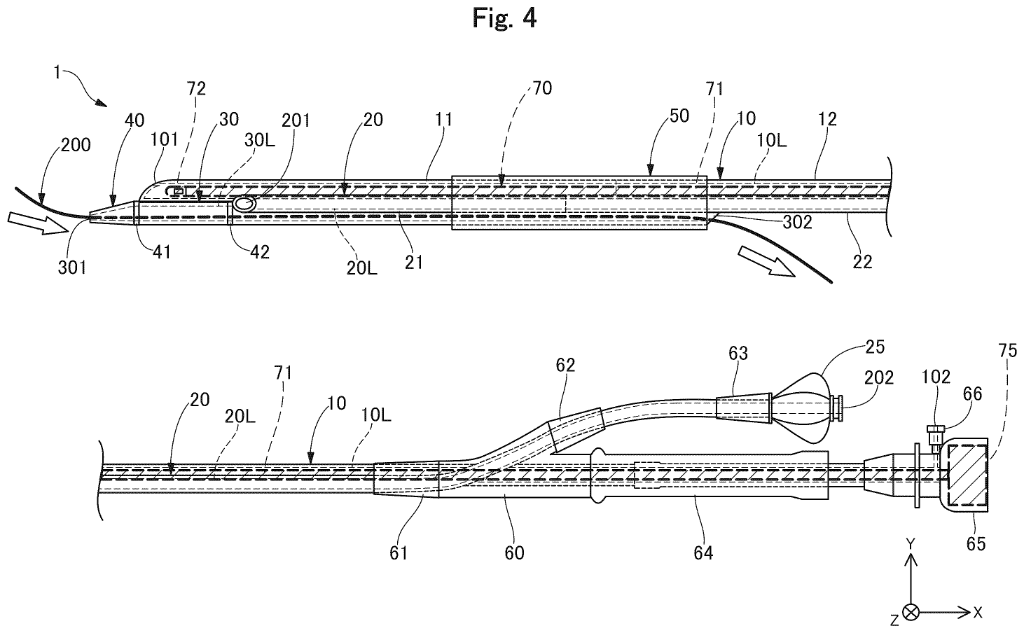

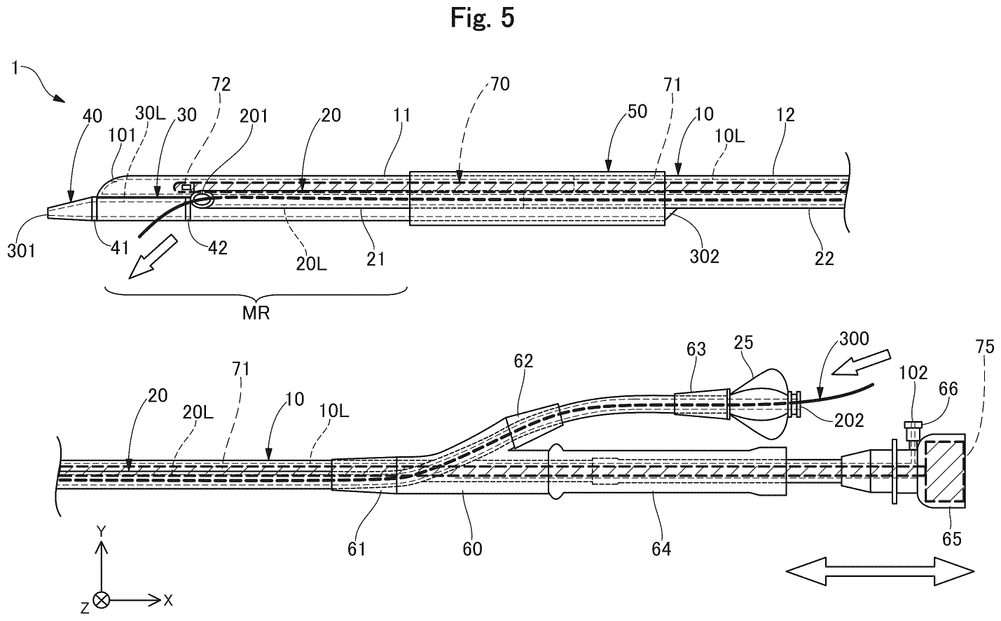

FIGS. 4 and 5 are diagrams describing a method of using the catheter. The following procedures a1 to a6 illustrate a case where a CTO (lesion) that has occurred in a blood vessel is recanalized using an antegrade approach. However, the catheter 1 may be used in a retrograde approach, and may be used for procedures other than CTO recanalization.

(a1) The operator inserts a workhorse wire 200 into the blood vessel, and delivers the distal end portion of the workhorse wire 200 near the CTO. (a2) The operator inserts the proximal end portion of the workhorse wire 200 from the distal end opening 301 of the catheter 1, and causes the proximal end portion of the workhorse wire 200 to pass through the RX lumen 30L, and be drawn out from the proximal end opening 302 of the catheter 1 (FIG. 4). (a3) The operator pushes the catheter 1 along the workhorse wire 200 into the blood vessel, and delivers the distal end portion of the catheter 1 near the CTO. In procedure a3, the catheter 1 may be delivered near the CTO by being passed through a guiding catheter previously inserted into the blood vessel along the workhorse wire 200. (a4) The operator grips and slides the connector 65 in the front-rear direction (FIG. 5: direction of white arrow) to adjust the position of the probe 72 of the sensor 70 within the range MR, and while checking the image displayed on the console terminal, aligns the position and orientation of the CTO and the distal end opening 201. The position refers to the position in the extending direction of the blood vessel, and the orientation refers to the orientation in the circumferential direction of the inner wall of the blood vessel. (a5) The operator inserts the distal end portion of a therapeutic device 300 from the proximal end opening 202 of the catheter 1, and causes the distal end portion of the therapeutic device 300 to be inserted through the OTW lumen 20L and protrude from the distal end opening 201 of the catheter 1 (FIG. 5). (a6) The operator treats the CTO using the therapeutic device 300 while adjusting the position of the probe 72 of the sensor 70 within the range MR as necessary, and checking the image displayed on the console terminal. As described above, an arbitrary device such as a plasma guidewire or a penetration guidewire can be used as the therapeutic device 300.

The sensor tube 10, the OTW tube 20, and the RX tube 30 are also collectively referred to as a “shaft”. The distal end side tube 11 and the distal end side tube 21 correspond to a “first tube”, and the proximal end side tube 12 and the proximal end side tube 22 correspond to a “second tube”. Note that, in the present embodiment, “same” and “equal” are not limited to strictly identical cases, and allows for differences due to manufacturing error and the like. Also, “constant” has the same meaning as “substantially constant”, and means being generally constant while allowing for deviations due to manufacturing error and the like.

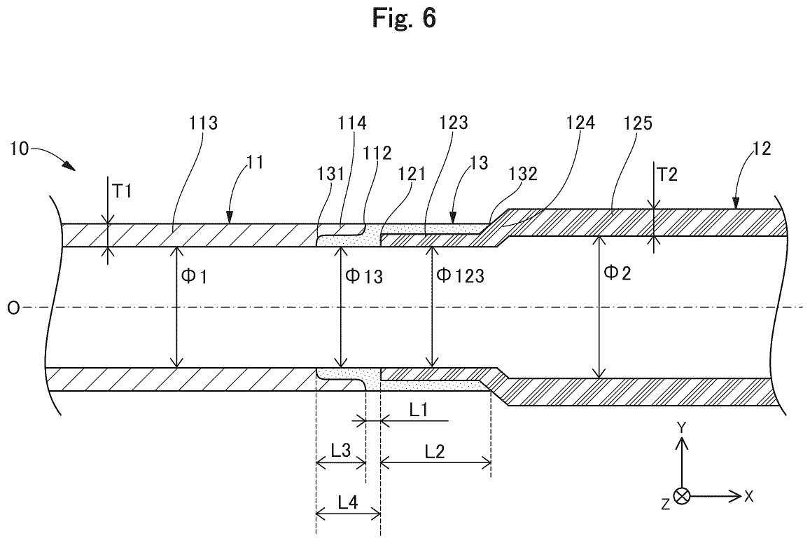

FIG. 6 is an enlarged longitudinal cross-sectional view of a part of a sensor tube 10. The details of the sensor tube 10 omitted from FIG. 1 will be described with reference to FIG. 6. In the sensor tube 10, a distal end side tube 11 (first tube) and a proximal end side tube 12 (second tube) are indirectly connected with a separation due to the connection member 13.

The distal end side tube 11 (first tube) has a main body portion 113 and a thin-walled portion 114. The main body portion 113 has a constant wall thickness T1 and a constant inner diameter Φ1. The thin-walled portion 114 is a part having a wall thickness thinner than the wall thickness T1 of the main body portion 113, and is provided further toward the proximal end side than the main body portion 113, or in other words, at the proximal end portion of the distal end side tube 11. As shown in the drawing, the thin-walled portion 114 maintains the same outer diameter as the main body portion 113, and is made thinner from the inner peripheral surface.

The proximal end side tube 12 (second tube) has a small-diameter portion 123, a tapered portion 124, and a large-diameter portion 125. The small-diameter portion 123 is a part where the outer diameter and inner diameter of the proximal end side tube 12 are each the smallest, and is provided on the distal end side of the proximal end side tube 12. The tapered portion 124 is a part where the outer diameter and inner diameter of the proximal end side tube 12 gradually increase from the distal end side toward the proximal end side. The tapered portion 124 is provided further toward the proximal end side than the small-diameter portion 123, or in other words, between the small-diameter portion 123 and the large-diameter portion 125. The large-diameter portion 125 is a part where the outer diameter and inner diameter of the proximal end side tube 12 are each the largest, and is provided further toward the proximal end side than the tapered portion 124, or in other words, on the proximal end side of the proximal end side tube 12. The large-diameter portion 125 has a constant wall thickness T2 and a constant inner diameter Φ2. As shown in FIG. 6, the distal end 121 of the proximal end side tube 12 is positioned further toward the proximal end side than the proximal end 112 of the distal end side tube 11.

Here, the wall thickness T2 of the large-diameter portion 125 of the proximal end side tube 12 is thicker than the wall thickness T1 of the main body portion 113 of the distal end side tube 11 (T2>T1). In a case where the large-diameter portion 125 or the main body portion 113 has a non-uniform wall thickness, the maximum value of the wall thickness is used for the wall thickness T2 of the large-diameter portion 125 and the wall thickness T1 of the main body portion 113. In addition, the inner diameter Φ2 of the large-diameter portion 125 of the proximal end side tube 12 is larger than the inner diameter Φ1 of the main body portion 113 of the distal end side tube 11 (Φ2>¢1). In a case where the large-diameter portion 125 or the main body portion 113 has a non-uniform inner diameter, the maximum value of the inner diameter is used for the inner diameter Φ2 of the large-diameter portion 125 and the inner diameter Φ1 of the main body portion 113. Note that, in the illustrated example, the inner diameter Φ13 of the connection member 13 and the inner diameter Φ123 of the small-diameter portion 123 of the proximal end side tube 12 are made to have the same size as the inner diameter Φ1 of the main body portion 113 of the distal end side tube 11.

The connection member 13 is a member that connects the distal end side tube 11 and the proximal end side tube 12. The connection member 13 makes contact with the distal end side tube 11 on the distal end side, and makes contact with the proximal end side tube 12 on the proximal end side. The inner surfaces of the distal end side tube 11, the connection member 13, and the proximal end side tube 12 align to collectively form the wall of a continuous lumen. In this way, the connection member 13 bridges the gap between the two tubes to provide a fluidly continuous path, allowing for the smooth passage of fluids, guidewires, or other medical instruments through the sensor lumen 10L The connection member 13 indirectly connects the distal end side tube 11 and the proximal end side tube 12 in a state where the distal end side tube 11 and the proximal end side tube 12 are separated, or in other words, in a state where the distal end side tube 11 and the proximal end side tube 12 are apart. That is, the distal end side tube 11 does not make contact with the proximal end side tube 12.

As shown in FIG. 6, the distal end side of the connection member 13 is integrated with the thin-walled portion 114 of the distal end side tube 11. For this reason, the distal end 131 of the connection member 13 is positioned further toward the distal end side than the proximal end 112 of the distal end side tube 11 (first tube). On the other hand, the proximal end side of the connection member 13 is integrated with the small-diameter portion 123 of the proximal end side tube 12. For this reason, the proximal end 132 of the connection member 13 is positioned further toward the proximal end side than the distal end 121 of the proximal end side tube 12 (second tube).

Here, let L1 be the axial direction distance from the proximal end 112 of the distal end side tube 11 (first tube) to the distal end 121 of the proximal end side tube 12 (second tube). Let L2 be the axial direction distance from the distal end 121 of the proximal end side tube 12 to the proximal end 132 of the connection member 13. Let L3 be the axial direction distance from the distal end 131 of the connection member 13 to the proximal end 112 of the distal end side tube 11. Let L4 be the axial direction distance of the inner peripheral surface of the connection member 13. The axial direction distance L4 can also be referred to as the axial direction distance of the part where the connection member 13 is exposed on the inner peripheral surface of the sensor tube 10. The “axial direction distance” is a straight-line distance along the central axis O of the sensor tube 10, or in other words, can also be considered a straight-line distance along the longitudinal direction of the sensor tube 10, or a straight-line distance along the X-axis direction in FIG. 6.

At this time, in the example of FIG. 6, the axial direction distance L1 is shorter than the total value of the axial direction distance L2 and the axial direction distance L3 (L1<L2+L3). Furthermore, the axial direction distance L1 is shorter than at least one of the axial direction distance L2 and the axial direction distance L3. In the illustrated example, the axial direction distance L1 is shorter than the axial direction distance L2 and also shorter than the axial direction distance L3 (L1<L2 and L1<L3). Also, the axial direction distance L4 is longer than the axial direction distance L1 (L4>L1).

The distal end side tube 11 (first tube) of the present embodiment is, for example, a three-layer tube having an inner layer, a middle layer, and an outer layer composed of different thermoplastic resins. The melting point of the distal end side tube 11 is referred to as a “first temperature”. Furthermore, the proximal end side tube 12 (second tube) of the present embodiment is, for example, a PEEK tube. The melting point of the proximal end side tube 12 is referred to as a “second temperature”. At this time, the first temperature and the second temperature are different temperatures, and the first temperature is lower than the second temperature (first temperature<second temperature). In the case of a tube composed of a plurality of layers of different materials, such as the distal end side tube 11 described above, the highest melting point among the plurality of layers is used as the “first temperature” or “second temperature”.

The connection member 13 of the present embodiment is, for example, a heat shrink tube formed of a fluororesin (PTFE or PFA). For example, the first tube may be a thermoplastic resin, the second tube may be a polyether ether ketone (PEEK) resin, and the connection member may be a polytetrafluoroethylene (PTFE) fluororesin. The melting point of the connection member 13 is referred to as a third temperature. At this time, the third temperature is higher than the lower of the first temperature and the second temperature. In the present embodiment, because first temperature<second temperature, when the melting point of the connection member 13 is added, the relationship becomes as follows: first temperature<third temperature<second temperature.

Such a sensor tube 10 can be prepared, for example, by the following procedures b1 to b5. In the following example, for convenience of the description, the material of the distal end side tube 11 is referred to as a “three-layer tube”, the material of the proximal end side tube 12 is referred to as a “PEEK tube”, and the material of the connection member 13 is referred to as a “PTFE tube”. However, the materials of the distal end side tube 11, the proximal end side tube 12, and the connection member 13 are not limited to the illustrated examples. The melting point of the three-layer tube is the same first temperature as the distal end side tube 11, the melting point of the PEEK tube is the same second temperature as the proximal end side tube 12, and the melting point of the PTFE tube is the same third temperature as the connection member 13.
(b1) The distal end side of the PEEK tube is processed to form the small-diameter portion 123 and the tapered portion 124 described in FIG. 6.
(b2) In a state where a core wire has been inserted, a cylindrical PTFE tube is placed over the distal end side of the PEEK tube (specifically, the part of the small-diameter portion 123 and the tapered portion 124 formed in procedure b1).
(b3) The entire PEEK tube and PTFE tube are heated with a heat gun or the like. The PEEK tube and the PTFE tube undergo heat shrinkage due to the heating.
(b4) After flaring the proximal end portion of the three-layer tube (a process that expands the proximal end portion and increases the diameter), the proximal end portion of the three-layer tube is placed over the distal end portion of the PTFE tube (the PTFE tube integrated with the PEEK tube in procedure b3).
(b5) The entire three-layer tube, PTFE tube, and PEEK tube are heated with a heat gun or the like. As a result of the heating, the three-layer tube and the PEEK tube are integrated via the PTFE tube, and the sensor tube 10, which consists of the distal end side tube 11, the connection member 13, and the proximal end side tube 12 having the structure described in FIG. 6, is formed.

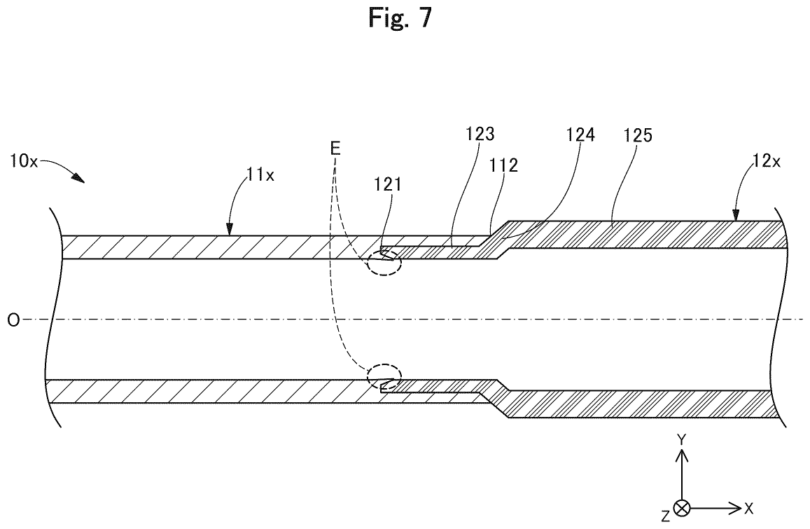

FIG. 7 is an enlarged longitudinal cross-sectional view of a part of a sensor tube 10x according to a comparative example. The sensor tube 10x of the comparative example, unlike the sensor tube 10 described in FIG. 6, has a distal end side tube 11x and a proximal end side tube 12x that are directly connected without the connection member 13 therebetween. After performing procedure b1 described above, the sensor tube 10x of the comparative example can be produced in procedure b2, in a state where a core wire has been inserted, by placing the three-layer tube over the distal end side of the PEEK tube (specifically, the part of the small-diameter portion 123 and the tapered portion 124 formed in procedure b1), and then heating the entire PEEK tube and three-layer tube. In the comparative example, procedures b3 to b5 are not performed. In such a sensor tube 10x of the comparative example, because the difference between the melting point of the three-layer tube (first temperature) and the melting point of the PEEK tube (second temperature) is large, when the entire PEEK tube and three-layer tube are heated, the three-layer tube melts before the PEEK tube undergoes shrinkage. As a result, in the sensor tube 10x of the comparative example, the distal end side tube 11x enters the inner peripheral surface side of the proximal end side tube 12x, causing an internal burr E to form. Such an internal burr E protrudes toward the inner peripheral surface side when the sensor tube 10x is bent, causing the additional device inside the sensor lumen 10L to get caught. Furthermore, even in a case where the sensor tube 10x is not bent, the internal burr E can cause the additional device to get caught depending on the shape of the additional device.

In this regard, the sensor tube 10 of the present embodiment shown in FIG. 6 uses a PTFE tube whose melting point (third temperature) is higher than the lower of the first temperature and the second temperature, and undergoes procedure b3 (the step of causing the PEEK tube and the PTFE tube to undergo heat shrinkage) described above. Therefore, it is possible to suppress the phenomenon that occurred in the comparative example, where the three-layer tube melts before the PEEK tube undergoes shrinkage, causing an internal burr E to form at the boundary between the PEEK tube and the three-layer tube. In addition, because the sensor tube 10 of the present embodiment integrates the distal end side tube 11, the proximal end side tube 12, and the connection member 13 using heat, compared to a case where the distal end side tube 11 and the proximal end side tube 12 are bonded using an adhesive, the occurrence of the phenomenon where the flexural rigidity locally increases at the part where the adhesive has set can be suppressed.

In FIG. 6, the sensor tube 10 has been described. However, the OTW tube 20 also has the same configuration as the sensor tube 10. That is, in the OTW tube 20, the distal end side tube 21 (first tube) and the proximal end side tube 22 (second tube) are indirectly connected with a separation due to a connection member. Moreover, the first temperature, which is the melting point of the distal end side tube 21, the second temperature, which is the melting point of the proximal end side tube 22, and the third temperature, which is the melting point of the connection member, have the same magnitude relationship as in the sensor tube 10. Also, the axial direction distances L1 to L4 of each portion of the distal end side tube 21 and the proximal end side tube 22 are the same as those of the sensor tube 10. The wall thicknesses and inner diameters of the distal end side tube 21 and the proximal end side tube 22 are also the same.

Note that the sensor tube 10 and the OTW tube 20 are tubes constituting the catheter 1. However, the sensor tube 10 may be used independently as a “medical tube”, or in a state where a connector or the like is attached to the proximal end portion.

As described above, in the catheter 1 (medical device) of the first embodiment, the distal end side tube 11 (first tube) and proximal end side tube 12 (second tube), which are disposed in the axial direction, are indirectly connected with a separation due to the connection member 13. Because the melting point of the connection member 13 is the third temperature, which is higher than the lower of the melting point of the distal end side tube 11 (first temperature) and the melting point of the proximal end side tube 12 (second temperature), the occurrence of an internal burr in the sensor tube 10 can be suppressed. In other words, according to the sensor tube 10 of the first embodiment, compared to a case where the distal end side tube 11x and the proximal end side tube 12x with a large difference in melting points are disposed without a connection member (the sensor tube 10x of the comparative example described in FIG. 7), the occurrence of the internal burr E can be suppressed. As a result, according to the sensor tube 10 of the first embodiment, the occurrence of an internal burr at the connecting part between two tubes (distal end side tube 11, proximal end side tube 12) disposed in the axial direction can be suppressed, thereby ensuring the integrity of a smooth and continuous fluid path through the lumen. The same points also apply to the OTW tube 20. Therefore, during use of the catheter 1, the additional device (sensor 70) may be prevented from getting caught in the sensor lumen 10L of the sensor tube 10, and the additional device (therapeutic device 300) from getting caught in the OTW lumen 20L of the OTW tube 20, and to suppress the procedure from being hindered due to catching, or the sensor tube 10 or OTW tube 20 from being damaged due to the catching.

Furthermore, in the catheter 1 (medical device) of the first embodiment, in the sensor tube 10, because the distal end 131 of the connection member 13 is positioned further toward the distal end side than the proximal end 112 of the distal end side tube 11 (first tube), the contact area between the connection member 13 and the distal end side tube 11 can be increased. In addition, because the proximal end 132 of the connection member 13 is positioned further toward the proximal end side than the distal end 121 of the proximal end side tube 12 (second tube), the contact area between the connection member 13 and the proximal end side tube 12 can be increased.

Furthermore, in the catheter 1 (medical device) of the first embodiment, in the sensor tube 10, because the axial direction distance L1 from the proximal end 112 of the distal end side tube 11 (first tube) to the distal end 121 of the proximal end side tube 12 (second tube) is shorter than the total value of the axial direction distance L2 from the distal end 121 of the proximal end side tube 12 to the proximal end 132 of the connection member 13, and the axial direction distance L3 from the distal end 131 of the connection member 13 to the proximal end 112 of the distal end side tube 11, the rigidity gap in the axial direction of the sensor tube 10 can be mitigated.

Moreover, in the catheter 1 (medical device) of the first embodiment, in the sensor tube 10, because the axial direction distance L1 from the proximal end 112 of the distal end side tube 11 (first tube) to the distal end 121 of the proximal end side tube 12 (second tube) is shorter than at least one of the axial direction distance L2 from the distal end 121 of the proximal end side tube 12 to the proximal end 132 of the connection member 13, and the axial direction distance L3 from the distal end 131 of the connection member 13 to the proximal end 112 of the distal end side tube 11, the rigidity gap in the axial direction of the sensor tube 10 can be mitigated.

In addition, in the catheter 1 (medical device) of the first embodiment, in the sensor tube 10, because the connection member 13 is formed of a fluororesin, and the axial direction distance L4 of the inner peripheral surface of the connection member 13 is longer than the axial direction distance L1 from the proximal end 112 of the distal end side tube 11 (first tube) to the distal end 121 of the proximal end side tube 12 (second tube), the slidability of the additional device on the inner peripheral surface of the connection member 13 can be improved.

Also, in the catheter 1 (medical device) of the first embodiment, in the sensor tube 10, because the maximum value Φ2 of the inner diameter of the proximal end side tube 12 (second tube) is larger than the maximum value Φ1 of the inner diameter of the distal end side tube 11 (first tube), the diameter of the sensor lumen 10L formed by the distal end side tube 11, the proximal end side tube 12, and the connection member 13 can be made to have a size that follows the changes in the outer diameter of a typical additional device. Furthermore, because the wall thickness T2 of the proximal end side tube 12 is thicker than the wall thickness T1 of the distal end side tube 11, the proximal end side of the sensor tube 10 can be made to have a higher rigidity compared to the distal end side.

In addition, because the catheter 1 (medical device) of the first embodiment is configured as a catheter 1 in which the sensor 70 that acquires image information is inserted into the sensor lumen 10L formed by the distal end side tube 11 (first tube), the proximal end side tube 12 (second tube), and the connection member 13, a procedure using image information (ultrasound images) acquired from the sensor 70 can be realized.

### Second Embodiment

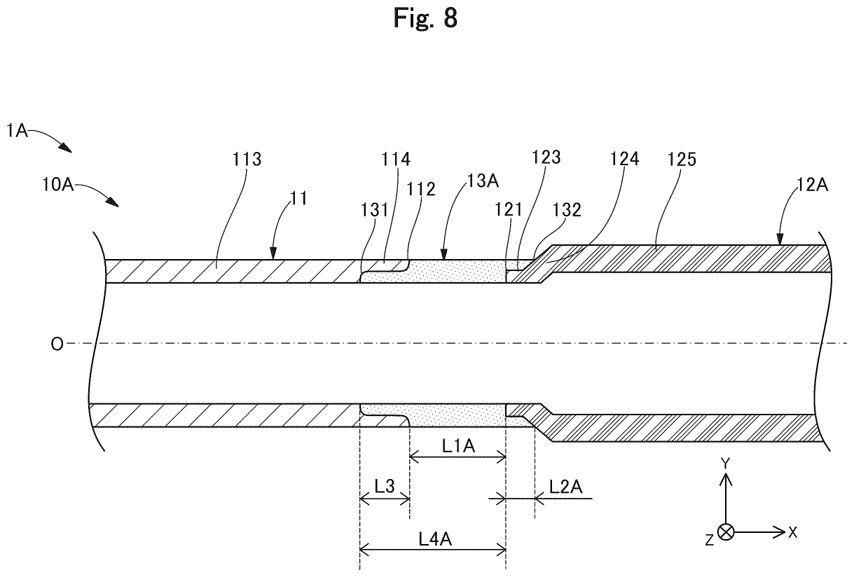

FIG. 8 is an enlarged longitudinal cross-sectional view of a part of a sensor tube 10A according to a second embodiment. The catheter 1A of the second embodiment has the configuration described in the first embodiment, but includes the sensor tube 10A instead of the sensor tube 10. The sensor tube 10A has the configuration described in the first embodiment, but includes a proximal end side tube 12A instead of the proximal end side tube 12, and a connection member 13A instead of the connection member 13.

The proximal end side tube 12A has a shorter length in the axial direction of the small-diameter portion 123 compared to the configuration of the first embodiment (FIG. 6). The connection member 13A has a longer length in the axial direction compared to the configuration of the first embodiment (FIG. 6). Here, let L1A be the axial direction distance from the proximal end 112 of the distal end side tube 11 (first tube) to the distal end 121 of the proximal end side tube 12A (second tube). Let L2A be the axial direction distance from the distal end 121 of the proximal end side tube 12A to the proximal end 132 of the connection member 13A. Let L3 be the axial direction distance from the distal end 131 of the connection member 13A to the proximal end 112 of the distal end side tube 11. Let L4A be the axial direction distance of the inner peripheral surface of the connection member 13A. At this time, the axial direction distance L1A is longer than the total value of the axial direction distance L2A and the axial direction distance L3 (L1A>L2A+L3). Furthermore, the axial direction distance L1A is longer than the axial direction distance L2A, and longer than the axial direction distance L3 (L1A>L2A and L1A>L3). In addition, the axial direction distance L4A is longer than the axial direction distance L1A (L4A>L1A).

In this way, in the sensor tube 10A, the axial direction distances L1A, L2A, L3, L4A, and the magnitude relationships between the distances can be arbitrarily changed. With the sensor tube 10A and the catheter 1A of the second embodiment as described above, the same effects as in the first embodiment can be achieved.

### Third Embodiment

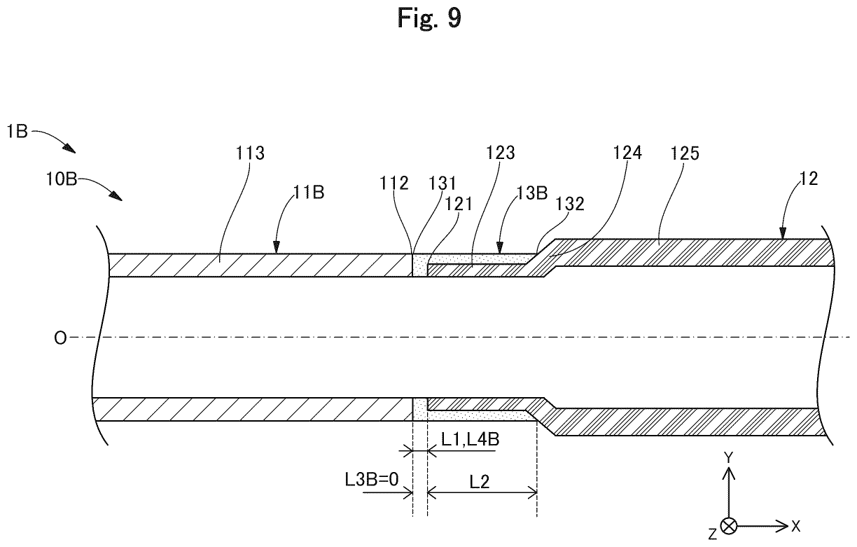

FIG. 9 is an enlarged longitudinal cross-sectional view of a part of a sensor tube 10B according to a third embodiment. The catheter 1B of the third embodiment has the configuration described in the first embodiment, but includes the sensor tube 10B instead of the sensor tube 10. The sensor tube 10B has the configuration described in the first embodiment, but includes a distal end side tube 11B instead of the distal end side tube 11, and a connection member 13B instead of the connection member 13.

The distal end side tube 11B does not have the thin-walled portion 114 described in the first embodiment (FIG. 6). Accordingly, the distal end side of the connection member 13B does not have a part that is integrated with the thin-walled portion 114. That is, in the third embodiment, the distal end 131 of the connection member 13B is at the same position as the proximal end 112 of the distal end side tube 11B (first tube).

Here, let L1 be the axial direction distance from the proximal end 112 of the distal end side tube 11B (first tube) to the distal end 121 of the proximal end side tube 12 (second tube). Let L2 be the axial direction distance from the distal end 121 of the proximal end side tube 12 to the proximal end 132 of the connection member 13B. Let L3B be the axial direction distance from the distal end 131 of the connection member 13B to the proximal end 112 of the distal end side tube 11B. Let L4B be the axial direction distance of the inner peripheral surface of the connection member 13B. At this time, the axial direction distance L1 is shorter than the total value of the axial direction distance L2 and the axial direction distance L3B (L1<L2+L3B). Furthermore, the axial direction distance L1 is shorter than at least one of the axial direction distance L2 and the axial direction distance L3B. In the illustrated example, the axial direction distance L1 is shorter than the axial direction distance L2 (L1<L2). Note that, in the illustrated example, the axial direction distance L3B is zero (L3B=0). On the other hand, the axial direction distance L4B is equal to the axial direction distance L1 (L4B=L1).

In this way, in the sensor tube 10B, the axial direction distances L1, L2, L3B, L4B, and the magnitude relationships between the distances can be arbitrarily changed. With the sensor tube 10B and the catheter 1B of the third embodiment as described above, the same effects as in the first embodiment can be achieved.

### Fourth Embodiment

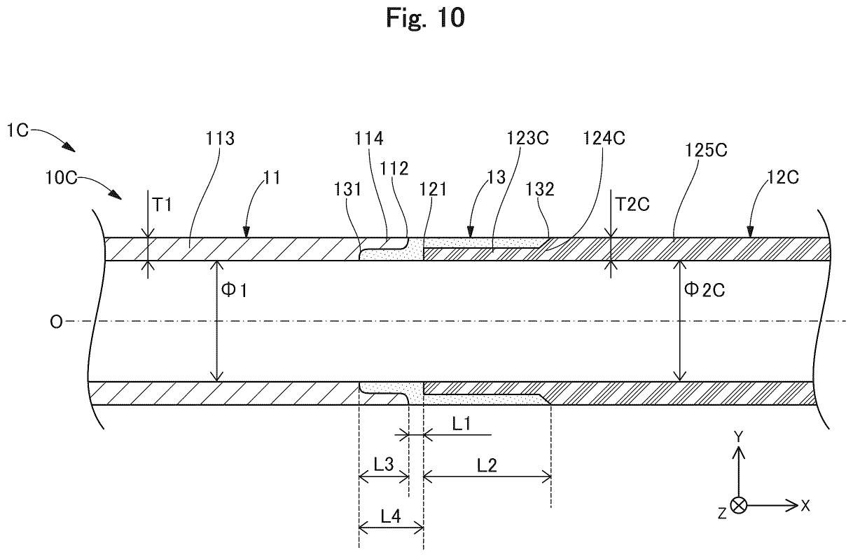

FIG. 10 is an enlarged longitudinal cross-sectional view of a part of a sensor tube 10C according to a fourth embodiment. The catheter 1C of the fourth embodiment has the configuration described in the first embodiment, but includes the sensor tube 10C instead of the sensor tube 10. The sensor tube 10A has the configuration described in the first embodiment, but includes a proximal end side tube 12C instead of the proximal end side tube 12.

The proximal end side tube 12C has a thin-walled portion 123C, a tapered portion 124C, and a main body portion 125C. The thin-walled portion 123C is a part having a wall thickness thinner than the wall thickness T2C of the main body portion 125C, and is provided on the distal end side of the proximal end side tube 12C. The tapered portion 124C is a part in which the outer diameter of the proximal end side tube 12C gradually increases from the distal end side toward the proximal end side. The tapered portion 124C is provided further toward the proximal end side than the thin-walled portion 123C, or in other words, between the thin-walled portion 123C and the main body portion 125C. The main body portion 125C is a part in which the outer diameter of the proximal end side tube 12C has the maximum diameter, and is provided further toward the proximal end side than the tapered portion 124C, or in other words, on the proximal end side of the proximal end side tube 12C. Here, the wall thickness T2C of the main body portion 125C of the proximal end side tube 12C is equal to the wall thickness T1 of the main body portion 113 of the distal end side tube 11 (T2C=T1). Also, the inner diameter Φ2C of the main body portion 125C of the proximal end side tube 12C is equal to the inner diameter Φ1 of the main body portion 113 of the distal end side tube 11 (Φ2C=Φ1).

In this way, in the sensor tube 10C, the maximum values of the wall thicknesses and inner diameters of the distal end side tube 11 and the proximal end side tube 12C can be arbitrarily changed. With the sensor tube 10C and the catheter 1C of the fourth embodiment as described above, the same effects as in the first embodiment can be achieved. Also, according to the sensor tube 10C of the fourth embodiment, the change in rigidity between the distal end side tube 11 and the proximal end side tube 12C can be made smaller.

### Fifth Embodiment

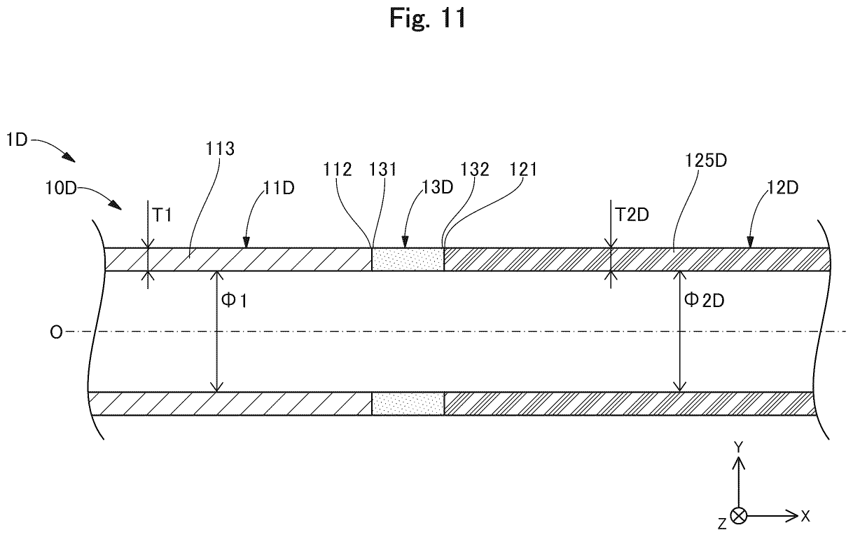

FIG. 11 is an enlarged longitudinal cross-sectional view of a part of a sensor tube 10D according to a fifth embodiment. The catheter 1D of the fifth embodiment has the configuration described in the first embodiment, but includes the sensor tube 10D instead of the sensor tube 10. The sensor tube 10D has the configuration described in the first embodiment, but includes a distal end side tube 11D instead of the distal end side tube 11, a proximal end side tube 12D instead of the proximal end side tube 12, and a connection member 13D instead of the connection member 13.

The distal end side tube 11D does not have the thin-walled portion 114 described in the first embodiment (FIG. 6). Accordingly, the distal end side of the connection member 13D does not have a part that is integrated with the thin-walled portion 114. That is, in the fifth embodiment, the distal end 131 of the connection member 13D is at the same position as the proximal end 112 of the distal end side tube 11D (first tube). The proximal end side tube 12D does not have the small-diameter portion 123 and the tapered portion 124 described in the first embodiment (FIG. 6), and has a main body portion 125D instead of the large-diameter portion 125. Accordingly, the proximal end side of the connection member 13D does not have a part that is integrated with the small-diameter portion 123. That is, in the fifth embodiment, the proximal end 132 of the connection member 13D is at the same position as the distal end 121 of the proximal end side tube 12D (second tube).

Furthermore, similarly to the fourth embodiment (FIG. 10), the wall thickness T2D of the main body portion 125D of the proximal end side tube 12D is equal to the wall thickness T1 of the main body portion 113 of the distal end side tube 11D (T2D=T1). In addition, the inner diameter Φ2D of the main body portion 125D of the proximal end side tube 12D is equal to the inner diameter Φ1 of the main body portion 113 of the distal end side tube 11D (Φ2D=Φ1).

In this way, in the sensor tube 10D, various modifications to the configurations of the distal end side tube 11D, the proximal end side tube 12D, and the connection member 13D are possible, and the positional relationship of the distal end 131 and the proximal end 132 of the connection member 13D with respect to the distal end side tube 11D and the proximal end side tube 12D can be arbitrarily changed. With the sensor tube 10D and the catheter 1D of the fifth embodiment as described above, the same effects as in the first embodiment can be achieved.

### Sixth Embodiment

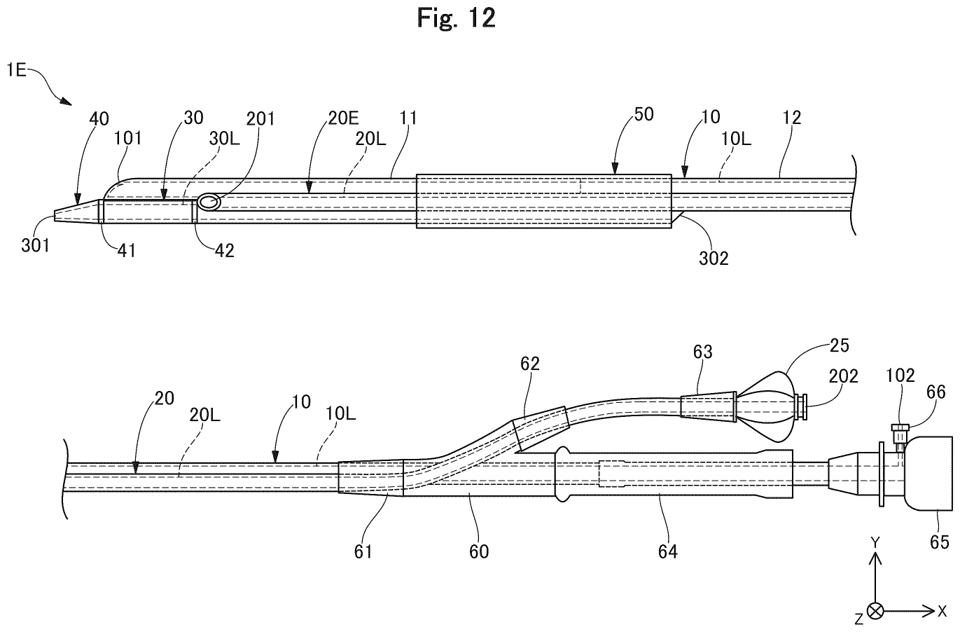

FIG. 12 is an explanatory diagram illustrating a configuration of a catheter 1E according to a sixth embodiment. The catheter 1E of the sixth embodiment has the configuration described in the first embodiment, but includes an OTW tube 20E instead of the OTW tube 20. The OTW tube 20E does not have the distal end side tube 21 and the proximal end side tube 22 described in the first embodiment (such as in FIGS. 1 and 6), and is configured by a single tube. In this way, various modifications to the configuration of the catheter 1E are possible, and only the sensor tube 10 may have the configuration described in FIG. 6. Furthermore, the sensor tube 10 may be configured by a single tube, and only the OTW tube 20E may have the configuration described in FIG. 6. With the catheter 1E of the sixth embodiment as described above, the same effects as in the first embodiment can be achieved.

### Seventh Embodiment

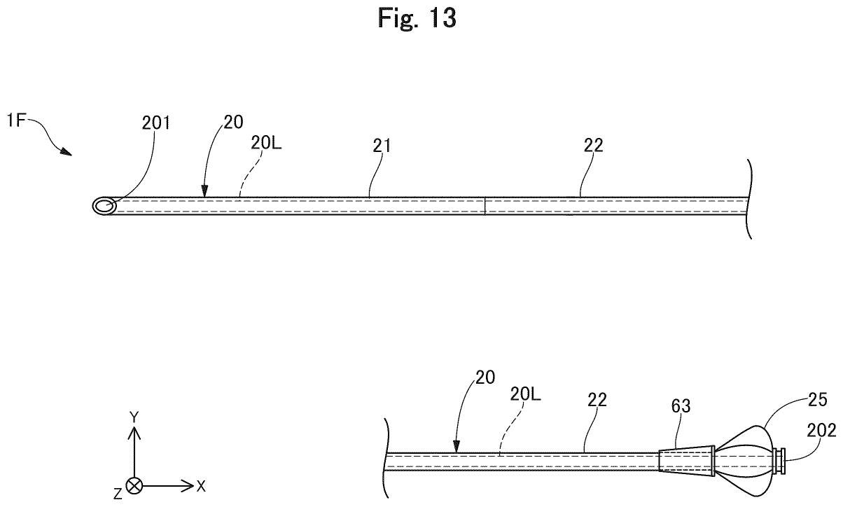

FIG. 13 is an explanatory diagram illustrating a configuration of a catheter 1F according to a seventh embodiment. The catheter 1F of the seventh embodiment includes an OTW tube 20, a third reinforcing member 63, and a connector 25. The OTW tube 20 has the same configuration as the sensor tube 10 described in FIG. 6. That is, in the OTW tube 20, the distal end side tube 21 (first tube) and the proximal end side tube 22 (second tube) are indirectly connected with a separation due to a connection member. Moreover, the first temperature, which is the melting point of the distal end side tube 21, the second temperature, which is the melting point of the proximal end side tube 22, and the third temperature, which is the melting point of the connection member, have the same magnitude relationship as in the sensor tube 10. Also, the axial direction distances L1 to L4 of each portion of the distal end side tube 21 and the proximal end side tube 22 are the same as those of the sensor tube 10. The wall thicknesses and inner diameters of the distal end side tube 21 and the proximal end side tube 22 are also the same. In this way, the OTW tube 20 may independently constitute a medical device (catheter 1F). With the catheter 1F of the seventh embodiment as described above, the same effects as in the first embodiment can be achieved.

### MODIFICATIONS OF EMBODIMENTS

The present disclosure is not limited to the embodiments described above, and can be implemented in various modes without departing from the spirit thereof, and for example, the following modifications are also possible.

### [Modification 1]

In the first to seventh embodiments described above, an example of the configuration of the catheters 1, and 1A to 1F has been described. However, various modifications can be made to the configuration of the catheters 1, and 1A to 1F.

For example, the outer peripheral surface of the covering portion 50, or the outer peripheral surface of the catheter 1 including the covering portion 50, may be coated with a hydrophilic resin or a hydrophobic resin. For example, the sensor 70 was built into the sensor lumen 10L of the sensor tube 10, and was configured so as to not be detachable from the catheter 1. However, the sensor 70 may be configured so as to be detachable from the catheter 1. That is, the catheter 1 does not have to include the sensor 70 as a component.

For example, at least one of the distal end tip 40, the first marker 41, and the second marker 42 may be omitted. For example, the shapes of the distal end tip 40, the first marker 41, and the second marker 42 can be arbitrarily changed. The distal end tip 40 may have a constant outer diameter from the distal end toward the proximal end, and the shape in the transverse cross-section may be a non-circular symmetric shape. The first marker 41 and the second marker 42 may have a shape that is different from an annular shape (for example, a shape obtained by cutting an annular shape at an arbitrary angle, a linear shape, or a coil shape in which a wire is spirally wound).

For example, the arrangement of the distal end tip 40, the first marker 41, and the second marker 42 can be arbitrarily changed. The first marker 41 may be disposed at a position that is different from a position adjacent to the proximal end of the distal end tip 40 (such as a position that is separated from the distal end tip 40). The second marker 42 may be disposed at a position that is different from a position adjacent to the distal end of the distal end opening 201 of the OTW tube 20 (such as a position that is separated from the distal end opening 201). The first marker 41 and the second marker 42 may be disposed on a tube different from the RX tube 30 (the sensor tube 10 or the OTW tube 20). The first marker 41 and the second marker 42 may be disposed on the same tube as described above, or may be disposed on different tubes.

For example, the covering portion 50 may be omitted. For example, in the example of FIGS. 3D and 3E, the covering portion 50 was composed of a single layer, but the covering portion 50 may be composed of a plurality of two or more layers. For example, in the example of FIGS. 3D and 3E, the covering portion 50 thinly covered the outer periphery of the three tubes 10, 20, and 30, thereby maintaining the recesses (concave portions) formed at the parts adjacent to each of the tubes 10, 20, and 30. However, the covering portion 50 may be configured so as to fill the recesses formed at the parts adjacent to each of the tubes 10, 20, and 30, such that the transverse cross-sectional shape of the catheter 1 becomes a circular shape or elliptical shape.

For example, the shapes of the branched connector 60, the first reinforcing member 61 to third reinforcing member 63, the cylindrical member 64, the connector 65, and the connector 25 described above are merely examples, and may be arbitrarily changed. For example, at least a part of the branched connector 60, the first reinforcing member 61, the second reinforcing member 62, and the cylindrical member 64 may be configured as a single member, or may be omitted. For example, the third reinforcing member 63 and the connector 25 may be configured as a single member. For example, the cylindrical member 64 may be provided with a mechanism that assists the adjustment of at least one of the front-rear position of the sensor 70 and the orientation of the sensor 70 in the circumferential direction (such as a scales or stoppers provided at predetermined lengths in the longitudinal direction, or scales or stoppers provided at predetermined angles in the circumferential direction).

### [Modification 2]

The configurations of the catheters 1, and 1A to 1F of the first to seventh embodiments and the configuration of the catheters 1, and 1A to 1F of Modification 1 described above may be combined as appropriate. The configuration of the sensor tube 10 described in the second to fifth embodiments may be incorporated into the catheter 1E described in the sixth embodiment, or incorporated into the catheter 1F described in the seventh embodiment.

### ASPECTS

(1) According to an aspect of the present disclosure, a medical device is provided. The medical device comprises: a first tube whose melting point is a first temperature; a second tube whose melting point is a second temperature, which is different from the first temperature, and whose distal end is positioned further toward a proximal end side than a proximal end of the first tube; and a connection member whose melting point is a third temperature, which is higher than the lower of the first temperature and the second temperature, the connection member being in contact with each of the first tube and the second tube, and connecting the first tube and the second tube in a separated state.

According to such a configuration, the occurrence of an internal burr in the medical device can be suppressed. Therefore, during use of the medical device, it is possible to suppress an additional device (for example, a workhorse wire, a therapeutic device, or a sensor) that has been inserted into the medical device from getting caught in the inner cavity of the medical device, and to suppress the procedure from being hindered due to the catching of the additional device, or the medical device from being damaged due to the catching of the additional device.

(2) In the medical device according to the above aspect, a distal end of the connection member may be positioned further toward a distal end side than the proximal end of the first tube, and a proximal end of the connection member may be positioned further toward the proximal end side than a distal end of the second tube. According to such a configuration, because the distal end of the connection member is positioned further toward the distal end side than the proximal end of the first tube, the contact area between the connection member and the first tube can be increased. Furthermore, because the proximal end of the connection member is positioned further toward the proximal end side than the distal end of the second tube, the contact area between the connection member and the second tube can be increased.

(3) In the medical device according to the above aspect, the connection member may be a heat shrink tube.

(4) In the medical device according to the above aspect, an axial direction distance L1 from the proximal end of the first tube to the distal end of the second tube may be shorter than a total value of an axial direction distance L2 from the distal end of the second tube to the proximal end of the connection member, and an axial direction distance L3 from the distal end of the connection member to the proximal end of the first tube. According to such a configuration, a rigidity gap in the axial direction of the medical device can be mitigated.

(5) In the medical device according to the above aspect, an axial direction distance L1 from the proximal end of the first tube to the distal end of the second tube may be shorter than at least one of the axial direction distance L2 from the distal end of the second tube to the proximal end of the connection member, and the axial direction distance L3 from the distal end of the connection member to the proximal end of the first tube. According to such a configuration, a rigidity gap in the axial direction of the medical device can be mitigated.

(6) In the medical device according to the above aspect, the connection member may be formed of a fluororesin, and an axial direction distance L4 of an inner peripheral surface of the connection member may be longer than the axial direction distance L1 from the proximal end of the first tube to the distal end of the second tube. According to such a configuration, the slidability of an additional device on the inner peripheral surface of the connection member can be improved.

(7) In the medical device according to the above aspect, a maximum value of an inner diameter of the second tube may be larger than a maximum value of an inner diameter of the first tube. According to such a configuration, the diameter of the lumen formed by the first tube, the second tube, and the connection member can be made to have a size that follows the outer diameter change of a typical additional device.

(8) In the medical device according to the above aspect, a wall thickness of the second tube may be thicker than a wall thickness of the first tube. According to such a configuration, the proximal end side of the medical device can be made more rigid compared to the distal end side.

(9) In the medical device according to the above aspect, a sensor that acquires image information may be inserted into a lumen formed by the first tube, the second tube, and the connection member. According to such a configuration, a procedure using image information (ultrasound images) acquired from the sensor can be realized.

Although the present aspect has been described above based on the embodiments and modifications, the embodiments of the aspect described above are for facilitating the understanding of the present aspect, and do not limit the present aspect. The present aspect can be modified or improved without departing from the spirit thereof and the scope of claims, and the present aspect also includes equivalents thereof. Furthermore, the technical features of the present aspects, if not indicated as essential herein, may be deleted as appropriate.

What is claimed is:

1. A medical device comprising:
a first tube having a melting point of a first temperature;
a second tube axially aligned with the first tube and having a melting point of a second temperature, which is different from the first temperature, wherein a distal end of the second tube is positioned proximal to a proximal end of the first tube; and
a connection member having a melting point of a third temperature, which is higher than the lower of the first temperature and the second temperature, the connection member being in contact with each of the first tube and the second tube, and connecting the first tube and the second tube while maintaining a separation between the proximal end of the first tube and the distal end of the second tube, wherein the connection member provides a continuous path between the first tube and the second tube.

2. The medical device according to claim 1, wherein
a distal end of the connection member is positioned further toward a distal end side than the proximal end of the first tube, and
a proximal end of the connection member is positioned further toward the proximal end side than a distal end of the second tube.

3. The medical device according to claim 1, wherein
the connection member is a heat shrink tube.

4. The medical device according to claim 1, wherein
a first axial direction distance from the proximal end of the first tube to the distal end of the second tube is shorter than a total value of a second axial direction distance from the distal end of the second tube to a proximal end of the connection member, and a third axial direction distance from a distal end of the connection member to the proximal end of the first tube.

5. The medical device according to claim 1, wherein
a first axial direction distance from the proximal end of the first tube to the distal end of the second tube is shorter than at least one of a second axial direction distance from the distal end of the second tube to the proximal end of the connection member, and a third axial direction distance from the distal end of the connection member to the proximal end of the first tube.

6. The medical device according to claim 1, wherein
the connection member is formed of a fluororesin, and
a fourth axial direction distance of an inner peripheral surface of the connection member is longer than a first axial direction distance from the proximal end of the first tube to the distal end of the second tube.

7. The medical device according to claim 1, wherein
a maximum value of an inner diameter of the second tube is larger than a maximum value of an inner diameter of the first tube.

8. The medical device according to claim 1, wherein
a wall thickness of the second tube is thicker than a wall thickness of the first tube.

9. The medical device according to claim 1, wherein
a sensor that acquires image information is inserted into a lumen formed by the first tube, the second tube, and the connection member.

10. The medical device according to claim 1, wherein the first temperature is lower than the second temperature, and the third temperature is greater than the first temperature and less than the second temperature.

11. The medical device according to claim 1, wherein an inner surface of the first tube, an inner surface of the connection member, and an inner surface of the second tube collectively form a substantially smooth and continuous inner wall of the lumen.

12. The medical device according to claim 1, wherein
a proximal end portion of the first tube includes a main portion and a thin-walled portion having a thickness less than a thickness of the main portion, and
the connection member overlaps with and is bonded to an outer surface of the thin-walled portion.

13. The medical device according to claim 1, wherein
a distal end portion of the second tube includes a first portion having a first diameter and a second portion having a second diameter smaller than the first diameter, and
the connection member overlaps with and is bonded to an outer surface of the second portion.

14. The medical device according to claim 1, wherein the medical device is a catheter.

15. The medical device according to claim 14, further comprising a third tube extending parallel to the first and second tubes, the third tube defining a third lumen.

16. A catheter comprising:
an elongate shaft having a proximal end and a distal end, the shaft including:
a sensor tube defining a sensor lumen, the sensor tube comprising: a distal tube section having a first melting temperature;
a proximal tube section having a second melting temperature different from the first melting temperature; and
a connection member having a third melting temperature that is higher than a lower of the first and second melting temperatures, the connection member fluidly coupling the distal tube section and the proximal tube section while maintaining the distal tube section and the proximal tube section in a separated, axially aligned configuration; and
an over-the-wire (OTW) tube extending parallel to the sensor tube and defining an OTW lumen.

17. The catheter according to claim 16, wherein the OTW tube includes a second distal tube section, a second proximal tube section, and a second connection member.

18. A method of manufacturing a medical tube, the method comprising:
providing a first tube having a proximal end and a melting point of a first temperature;
providing a second tube having a distal end and a melting point of a second temperature, the second temperature being different from the first temperature;
providing a connection member having a melting point of a third temperature, the third temperature being higher than a lower of the first temperature and the second temperature; assembling the first tube, the second tube, and the connection member such that the connection member bridges a gap between the proximal end of the first tube and the distal end of the second tube; and
heating the assembly to cause the connection member to thermally bond to both the first tube and the second tube.
